(19) United States
(12) Patent Application Publication
(10) Pub. No.: US 2023/0101488 A1
(43) Pub. Date: Mar. 30, 2023

(54) SURGICAL LASER FIBER WITH REFLECTIVE STANDOFF SLEEVE AND METHOD OF PREVENTING DUST PARTICLE BUILDUP WITHIN A STANDOFF SLEEVE

(71) Applicant: OPTICAL INTEGRITY, INC., PANAMA CITY BEACH, FL (US)

(72) Inventors: JOE D. BROWN, PANAMA CITY BEACH,, FL (US); HOWARD S. KLYMAS, PANAMA CITY BEACH,, FL (US); DANIEL MALPHURS, PANAMA CITY BEACH,, FL (US)

(21) Appl. No.: 17/950,414

(22) Filed: Sep. 22, 2022

Related U.S. Application Data
(60) Provisional application No. 63/324,676, filed on Mar. 29, 2022.
(60) Provisional application No. 63/247,427, filed on Sep. 23, 2021.

(51) Int. Cl. H01S 3/067; G02B 6/02

(52) U.S. Cl. CPC H01S 3/067; G02B 6/02; H01S 3/06708

(57) ABSTRACT

An end-firing surgical laser fiber suitable for Thulium Laser Fiber lithotripsy applications includes an internally reflective tube that extends beyond the distal end surface of the fiber to provide a standoff sleeve, and that is welded or otherwise fixed to an end section of the fiber. The standoff sleeve may be made of silica glass or sapphire, a reflective metal, and/or may include a reflectivity-enhancing coating or structure on an inner surface of the tube. In addition, the reflective standoff sleeve may be tapered to increase or decrease a diameter of a distal end of the sleeve to control output power density, and may include index matched fillers or structures that absorb, transmit, or scatter energy away from the fiber cladding, and/or an energy blocking or absorbing structure positioned at an upstream end of the sleeve. Still further, the laser output may be modified by adding relatively low power, extended duration pulses to a high frequency pulse train in order to clear suspended dust particles from an interior of the sleeve during a lithotripsy procedure, and prevent buildup of the particles on the inside diameter of the sleeve.

The application claims the benefit of U.S. Provisional Appl. Ser. No. 63/324,676, filed Mar. 29, 2022, and 63/247,427, filed Sep. 23, 2021, each of which is incorporated herein by reference.

### BACKGROUND OF THE INVENTION

### 1. Field of the Invention

The invention relates to a surgical laser fiber.

More particularly, the present invention relates to configuration of an end-firing surgical laser fiber, and in particular to an end-firing surgical laser fiber having a distal end section to which is fixed a sleeve or tube having a highly reflective inner diameter to maintain fiber output power density by acting as a waveguide for laser radiation exiting the fiber and incident on the inner wall of the sleeve or tube, and which further serves as a standoff sleeve to provide a minimum spacing between the end face of the fiber and a target of the surgical laser.

The invention also relates to a method of preventing accumulation of dust particles and stone fragments on inside diameter of a standoff sleeve.

The surgical laser fiber and method of the invention may be used with a variety of different laser types, including Thulium Fiber Lasers (TFLs) of the type used in laser lithotripsy (urological stone fragmentation) procedures.

### 2. Description of Related Art

Laser lithotripsy is a surgical procedure developed during the 1980s to remove impacted stones from the urinary tract, i.e., kidney, ureter, bladder, or urethra, by fragmenting or breaking apart the stones so that they can be more easily excreted by the patient. Early laser lithotripsy used pulsed-dye lasers with picosecond pulse durations to create cavitation bubbles whose collapse caused laser induced shockwaves to fragment the stone. However, the induced shockwave caused a high degree of retro-repulsion, i.e., pushing of the stone way from the laser delivery fiber, and therefore the pulse dye lasers were replaced by pulsed Holmium lasers having longer pulse durations (250 micro seconds) that produced a weaker pressure wave, and therefore less retro-repulsion.

Such laser lithotripsy procedures required frequent replacement of the laser delivery fibers due to fiber degradation. In addition, operators of the Holmium laser frequently encountered sudden flashes that temporarily prevented the operator from viewing the treatment site, forcing stoppage of the laser and prolonging the lithotripsy procedure. It was found that both problems could be traced back to free electron absorption (FEA) by the fiber-as a result of contact between the tip of the fiber and the stone being targeted, and therefore the current inventors proposed to eliminate the possibility of fiber-to-stone contact by providing a standoff sleeve that extended beyond the tip of the fiber to physically maintain a minimum spacing between the fiber and the stone. As described in the inventor's PCT Appl. Ser. No. PCT/US2017/031091 (PCT Publ. No. WO/2017/192869), filed May 4, 2017, the spacer tip or standoff sleeve consisted of a non-reflective cylindrical structure placed over a stripped end of the fiber, and arranged to extend a predetermined distance beyond the end to act as a physical barrier between the fiber and a stone. Not only did the standoff sleeve prevent contact between the fiber and the stone but, by making the sleeve out of a relatively soft material such as PTFE or ETFE, the standoff sleeve could be used to protect the inner surface of an endoscope during insertion of the fiber through the scope to the treatment site.

Further enhancements to the spacer tips or standoff sleeves, and/or methods of utilizing the spacer tips or sleeves during laser lithotripsy procedures included the arrangements and methods disclosed by the inventors in U.S. patent application Ser. No. 15/992,609, filed May 30, 2018, now U.S. Pat. No. 11,109,911; Ser. No. 16/234,690, filed Dec. 28, 2018; Ser. No. 16/353,225, filed Mar. 14, 2019; Ser. No. 16/414,255, filed May 16, 2019; and Ser. No. 16/546,992, filed Aug. 21, 2019. For example, it was found that by appropriately controlling the laser, or by use of an additional continuous wave laser, liquid in the interior passage between the end of the fiber and the end of the sleeve could be vaporized, resulting in a “Moses” effect that reduces retro-repulsion of the stone, resulting in reduced power requirements, enhanced stone fragmentation efficiency, and shorter treatment times.

However, while the above-described PTFE or ETFE spacer tips or standoff sleeves are well-suited for use with existing pulsed Holmium:YAG laser systems, problems arise when used in continuous wave/high frequency Thulium fiber lasers (TFLs), which have recently replaced pulsed Holmium:YAG lasers for many lithotripsy applications because of their relatively small Gaussian beam profile, which allows use of smaller fiber core diameters (improving fiber flexibility and irrigation in the single working channel of the scope), and because the lack of pulse intervals prevents fragments of stone from breaking away and escaping the path of the laser during the intervals. On the other hand, the smaller cross-section and higher power density of a TFL fiber leaves the fiber more vulnerable to degradation due to free electron absorption and increased temperatures at the treatment site. Thulium fiber lasers typically have an output frequency of 5-2500 Hz, as compared to 5-100 Hz for a pulsed Holmium laser, resulting in substantially increased heat generation at the treatment site, which can destroy spacer tips or standoff sleeves of the type described above, leaving no way to prevent fiber-to-stone contact.

One solution to reducing fiber degradation is simply to increase the size of the fiber, resulting in a more robust fiber that is able to withstand higher temperatures and to absorb reflected radiation, but this would negate the advantages of increased flexibility and enhanced irrigation resulting from the smaller fiber diameter made possible by the use of continuous wave Thulium fiber lasers. In order to maintain the advantages of a thinner fiber while at the same time reducing fiber degradation by providing a more robust fiber exit surface, it was proposed to provide an outward taper at the end of the fiber, for example as disclosed in copending U.S. patent application Ser. No. 15/417,934, filed Jan. 27, 2017 (BROW3039CIP). However, as discussed in Section 1.3 on page 2 of the article by Thomas C. Hutchens et al. entitled “Hollow Steel tips for Reducing Distal Fiber Burn-Back During Thulium Fiber Laser Lithotripsy,” Journal of Biomedical Optics, 18(7), 078001 (July 2013), the tapered fiber was still vulnerable to damage and burn-back, and furthermore more delicate and subject to fracture during handling. Consequently, the Hutchens article proposed to replace the tapered fiber tip with a hollow steel tube glued to, and arranged to surround and extend beyond, the end of a conventional non-tapered, cylindrical fiber.

Neither of these prior solutions to the problem of fiber degradation in a Thulium laser system completely addresses the problems of fiber degradation and maintaining spacing between the fiber tip and a target stone. As a result, there is still a need for fiber tip configurations or arrangements that can prevent fiber degradation in systems such as Thulium laser lithotripsy system that use higher power lasers and smaller diameter fibers.

In addition, the prior solutions to the problem of fiber degradation in any type of laser lithotripsy system that utilizes a standoff sleeve fail to address another cause of fiber degradation, namely degradation caused by a buildup of dust particles on the inside of the standoff sleeve.

While high power lasers, including continuous wave Thulium lasers, have the advantage of more completely pulverizing stone fragments, this can lead to the creation of additional suspended dust particles. If too many dust particles build up inside the sleeve, the temperature of the fiber tip can still exceed 1000° C. and create Free Electron Absorption (FEA) similar to the FEA that occurs when a fiber tip contacts a stone in the absence of a sleeve, resulting in rapid fiber degradation despite the standoff sleeve.

### SUMMARY OF THE INVENTION

It is accordingly an objective of the present invention to provide an improved optical fiber arrangement for laser surgery applications.

It is a second objective of the invention to provide a surgical laser system that takes advantage of the smaller fiber diameter made possible by the use of higher power density lasers, while still providing durability advantages of a larger diameter fiber, i.e., lower vulnerability to fiber degradation due caused by energy reflected or emitted by the target.

It is a third objective of the invention to provide an optical fiber arrangement for continuous wave laser lithotripsy applications, which further includes a standoff sleeve that prevents the fiber tip from contacting a stone, and that is capable of withstanding the higher treatment site temperatures generated by the continuous wave lasers, including relatively high power lasers such as Thulium fiber lasers having wavelengths of, for example, 1900 to 2200 nm.

It is a fourth objective of the invention to provide a surgical laser system having an optical fiber configured at the treatment end to offer both enhanced resistance to damage caused by higher treatment site temperatures, and a minimum spacing between the end of the fiber and a target of the laser to prevent fiber degradation due to FEA and other problems resulting from contact between the fiber tip and the stone.

It is a fifth objective of the invention to provide a method of preventing damage caused by dust particle buildup on the inside diameter of a standoff sleeve, by adding lower power single or multiple pulses to the laser output in order to flush suspended dust particles from the interior of the standoff sleeve.

These and other objectives of the invention are achieved, in accordance with the principles of an exemplary embodiment of the invention, by replacing the relatively soft PTFE or ETFE standoff sleeves utilized with conventional Holmium fiber lasers, and the hollow steel tube proposed in the Hutchens article cited above, with a standoff sleeve having a reflective inner diameter to maintain power density, and yet that is capable of withstanding the higher treatment temperatures of, for example, a high pulse frequency Thulium fiber laser.

The objectives of the invention are also achieved by providing a method of preventing buildup of dust particles within a standoff sleeve, whether conventional or modified to include a reflective inner diameter, by modifying the laser output to include low power long-duration single or multiple pulses that serve to flush suspended dust particles from the inside of the standoff sleeve. The particle-flushing pulses could be applied periodically, as a pre-pulse or in response to detection of excess radiation or FEA, for example by using the stone-sensing method described in copending U.S. patent application Ser. No. 15/992,609, filed May 30, 2018 (now U.S. Pat. No. 11,109,911), and Ser. No. 17/400,380, filed Aug. 12, 2021, both of which are incorporated herein by reference.

In exemplary embodiments of the invention, the internally-reflective standoff sleeve may be made of metal, a glass material such as silica glass, or sapphire. In addition or alternatively, the inner diameter of the sleeve may be provided with coatings to enhance reflectivity. Preferably, the standoff sleeve is fixed to the outer diameter of an end section of the fiber by welding, and the end section of the fiber to which the standoff sleeve is welded be tapered or non-tapered. If a taper is provided, the region around the circumference of the outwardly-expanded planar end face of the fiber is welded to the standoff sleeve, and the space between the tapered section and the sleeve may be filled with a filler material or reinforcing structure, such as an insert sleeve, so that the advantages of a tapered tip can be obtained without the disadvantages of fragility and susceptibility to fracture of the tapered joint. Welding of the standoff sleeve to the fiber provides the further advantage of providing a heat conductive connection that enable the sleeve to serve as a heat sink, although it is also within the scope of the invention to utilize other adhesive or mechanical methods (such as crimping) to fix the reflective standoff sleeve to the fiber, depending on the material of the reflective standoff sleeve. In addition, the filler material or reinforcing structure may be index matched to the material of the sleeve and have an index of refraction that matches, or is higher than, that of the cladding to absorb, transmit, or scatter energy that might otherwise back-propagate through the fiber towards the scope.

Although the reflective standoff sleeve is not limited to use with tapered fibers, tapering of the fiber provides the advantage of lowering the numerical aperture of the fiber to concentrate laser power, while index matching of the sleeve and fiber cladding prevents free electron absorption (FEA) radiation from traveling back down the fiber and causing fiber breakage within the scope through which the fiber has been inserted. In addition, the reflective standoff sleeve acts a heat sink to further help prevent damage due to free electron absorption, and as a waveguide for laser radiation incident on the inner wall of the sleeve. Although made of a relatively hard material, damage to the scope during insertion of the fiber may be prevented by rounding the distal end of the sleeve. Unlike a bare fiber which can erode exposing sharp tip edges, the sleeve does not erode and a smooth edge is maintained to prevent scope damage.

In a second exemplary embodiment of the invention, the end of the fiber is formed as or includes a convex lens structure to further focus the laser output of the fiber. It will be appreciated that the convex lens structure may include a variety of shapes, including planar, tapered, and/or curved shapes, and combinations thereof.

In addition to cylindrical standoff sleeve shapes, it will be appreciated that the shape of the reflective standoff sleeve may also be varied to increase or decrease power density output, by expanding or reducing the diameter of the distal or output end of the reflective standoff sleeve.

### BRIEF DESCRIPTION OF THE DRAWINGS

FIG. 1 is a cross-sectional side view of a laser lithotripsy standoff sleeve arrangement constructed in accordance with the principles of a first exemplary embodiment of the present invention.

FIG. 2 is a cross-sectional side view of a laser lithotripsy standoff sleeve arrangement constructed in accordance with the principles of a second exemplary embodiment of the invention.

FIG. 3 is a cross-sectional side view of a laser lithotripsy standoff sleeve arrangement constructed in accordance with the principles of a third exemplary embodiment of the invention.

FIGS. 4A and 4B are cross-sectional side views of alternative laser lithotripsy standoff sleeve arrangements constructed in accordance with the principles of a fourth exemplary embodiment of the invention.

FIG. 5 is a cross-sectional side view of a laser lithotripsy standoff sleeve arrangement constructed in accordance with the principles of a fifth exemplary embodiment of the invention.

FIGS. 6A and 6B illustrate the manner in which a laser output is controlled to flush suspended dust particles and prevent dust particle buildup on the inside of a standoff sleeve, according an exemplary method that may be used with the standoff sleeves shown in FIGS. 1-5.

### DETAILED DESCRIPTION OF THE PREFERRED EMBODIMENTS

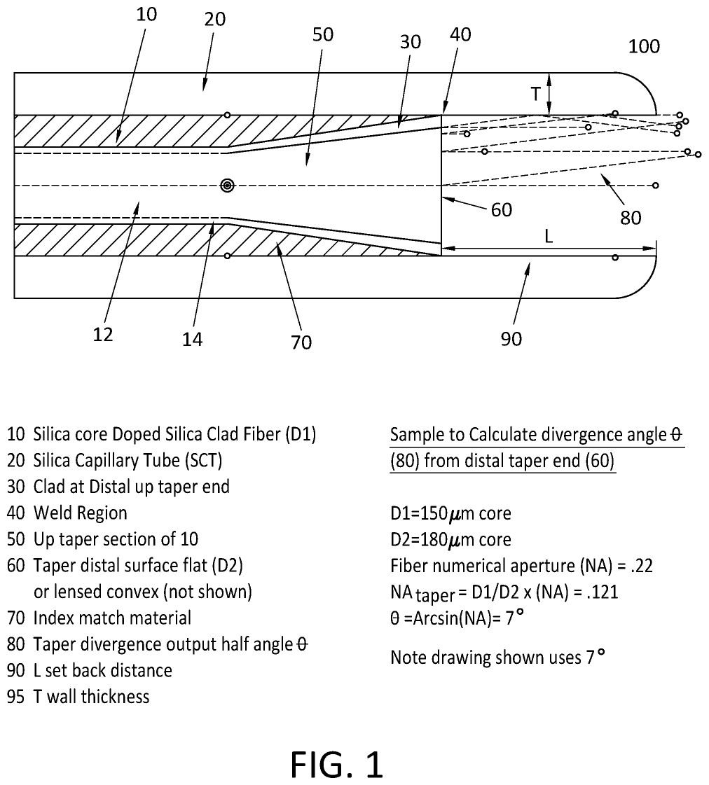

As shown in FIG. 1, a laser lithotripsy fiber tip arrangement constructed in accordance with principles of a preferred embodiment of the invention includes an optical fiber 10 that has been stripped to leave a core 12 and one or more cladding layers 12. The fiber, including the proximal end coupled to the laser and through which laser energy is injected into the fiber, may be conventional, with the exception as described below of the treatment end, i.e., the distal end of the fiber that is inserted through a scope (not shown) to the treatment site. In the embodiment of FIG. 1, the distal end of the fiber includes a flat surface 60 through which laser energy is directed at a targeted stone (not shown) to break it apart during a lithotripsy procedure, while the clinician or operator of the laser views the stone and the fiber end through the scope.

In the exemplary embodiment of FIG. 1, the fiber 10 is tapered outwardly to form a tapered section 50 of so that the flat distal end surface 60 has a diameter D2 that is larger than a diameter D1 of the non-tapered portion of the fiber. Preferably, the core 12 of the fiber in the tapered section 50 has a diameter that increases linearly in an axial direction from the start of the taper to the flat distal end surface 60. At least one cladding layer 14 of the fiber 10 extends over the tapered section 50, the extended cladding layer 30 also having, by way of example and not limitation, a linearly increasing thickness. Although the exemplary embodiment includes a tapered section, it will be appreciated that tapering is not required, and that the standoff sleeve 20 described below may be utilized with a non-tapered fiber.

For a Thulium Laser Fiber, exemplary dimensions are as follows: the core diameter D1 of the fiber 10 is 150 μm, and the diameter of the flat distal end surface 60 of the tapered section 50 may be 180 μm. For a fiber having a numerical aperture (NA) of 0.22, the numerical aperture of the taper is 0.121 (0.22 multiplied by the ratio of start and end diameters (D1/D2)). The divergence output half angle θ from the taper end, indicated by reference numeral 80 (θ=arcsin(NA)) is 7°.

It will be appreciated by those skilled in the art that the taper, if provided, can be achieved by known optical fiber formation methods, including appropriate control of core extrusion and cladding coating processes, and that the dimensions of the taper may be varied without departing from the scope of the invention.

The standoff sleeve 20 that surrounds the tapered end 60 of the fiber 10 in the exemplary embodiment of FIG. 1 has a reflective inner surface or diameter and is welded to an end section of the fiber 10 in a weld region 40 that extends at least partially or intermittently around a circumference of the fiber end surface 60. The reflectivity of the inner surface enables the sleeve to serve as a waveguide for radiation exiting the fiber, particularly at low divergence angles. Suitably reflective materials for the sleeve 20 include various metals, fused silica glass, and sapphire. In addition, or alternatively, reflective coatings may be provided on the inner surface of the sleeve to enhance reflectivity. As with conventional standoff sleeves, the inner diameter of the reflective standoff sleeve 20 can be used to maintain an air pocket or bubble inside the ferrule resulting from vaporization of the fluid during lasing, thereby limiting retro-repulsion of the targeted stone.

To prevent the end surface 60 from contacting a stone during a lithotripsy procedure, the fiber end surface 60 is set back from the distal end 100 of the standoff sleeve 20 by a set-back distance L. The standoff sleeve 20 has a thickness T and may, for example, be in the form of a silica capillary tube (SCT) 10 that is index matched to, or that has a refraction index that is higher than, the index of the fiber cladding. Such an all silica glass sleeve can handle higher temperatures than an ETFE or PTFE sleeve, while also acting as a heat sink to help prevent damage due to free electron absorption (FEA), and as a waveguide for laser radiation exiting the fiber. The distal end 100 of the standoff sleeve 20 may further be rounded to provide protection for a scope (not shown) through which the fiber 10 is inserted to the treatment site.

The space between the tapered section 50 of fiber 10 and the standoff sleeve 20 is preferably filled with an index-matching reinforcing filler material 70. Reinforcing filler material 70 preferably has an index of refraction that matches the index of refraction of the sleeve 20 and that is equal to or higher than that of the cladding 30, to facilitate dissipation of radiation reflected or emitted back into the fiber from the treatment site, so that the radiation does not travel back through the fiber and cause damage to the fiber. Although illustrated as being between the tapered section 50 and the standoff sleeve 20, the reinforcing filler material 70 may also be present between the non-tapered portion of the fiber 10 and the standoff sleeve 20.

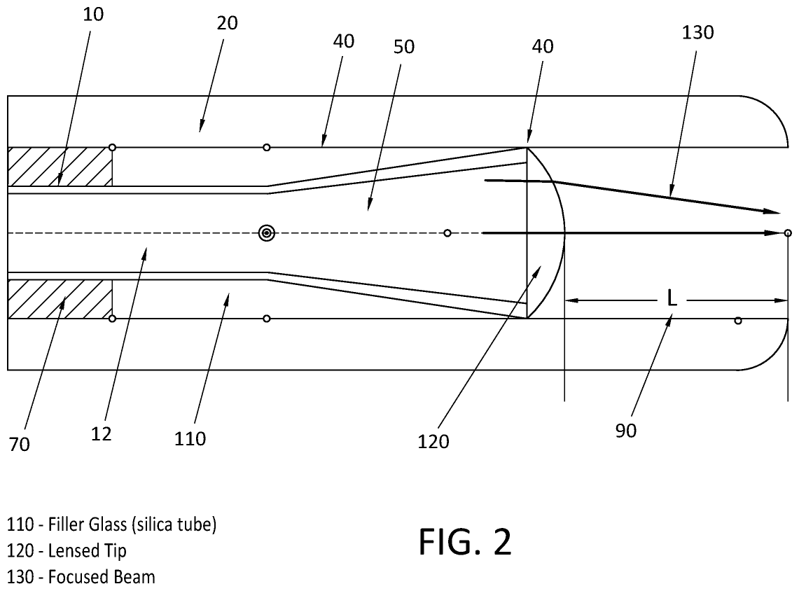

FIG. 2 shows a second exemplary embodiment of the invention, which adds a convex lens structure 120 that is adhered to the end face of the fiber, or formed by rounding the fiber tip, to further collimate or focus the beam 130 exiting the fiber. The tapered section 50 and standoff sleeve 20 of this embodiment may be otherwise identical to the corresponding tapered section and standoff sleeve 20 of FIG. 1.

FIG. 2 also shows an optional feature in which all or at least a portion of the index matching reinforcing filler material 70 is replaced by a silica tube 110 index matched to the silica glass sleeve 20. This optional feature may also be included in the fiber arrangement of the exemplary embodiment of FIG. 1. It will be appreciated that either the flat tip or the rounded or lensed tip may be included on a tapered fiber, as illustrated, or on a non-tapered fiber.

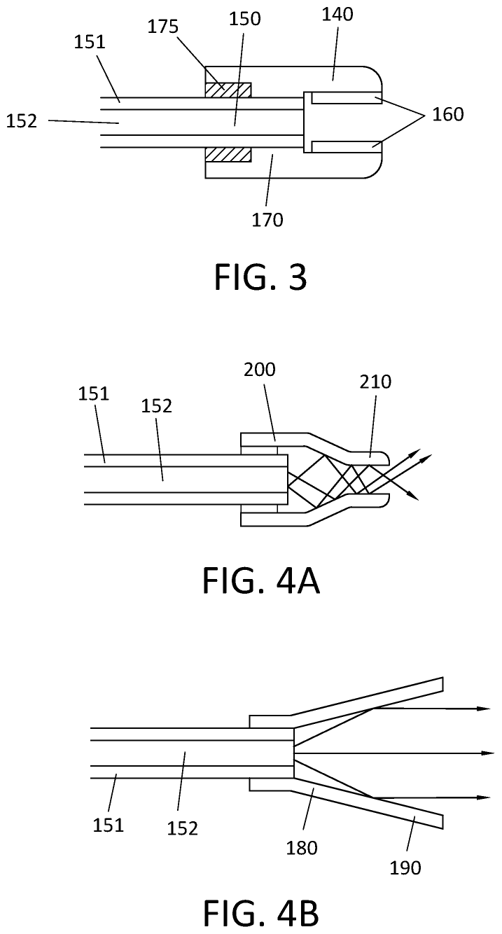

FIG. 3 shows a variation of the first exemplary embodiment in which the reflective standoff sleeve is formed from a cylindrical outer sleeve 140 that is fixed or welded to the cladding 151 surrounding core 152 of the fiber 150, and a reflective coating or insert 160. The outer sleeve 140 may be made of a material that can be welded to the cladding, and may optionally include an inwardly extending collar 170 to facilitate fixing or welding, or the outer sleeve 140 can be made of a softer material such as PTFE or ETFE. If a softer material is utilized, the reflective insert or coating 150 can be made of a heat resistant material to prevent heat damage to the outer sleeve 140. Finally, an index matched filler 174 may also be provided as in the embodiments of FIGS. 1 and 2.

In addition to cylindrical standoff sleeve shapes, it will be appreciated that the shape of the reflective standoff sleeve may also be varied to increase or decrease power density output, by expanding or reducing the diameter of the distal or output end of the reflective standoff sleeve as shown respectively in FIGS. 4A and 4B. FIG. 4A shows a tapered standoff sleeve 180 that has an expanded diameter distal or output end 190 to decrease the output power density, while FIG. 4B shows a tapered standoff sleeve 200 is swaged down to reduce the diameter of the distal or output end 210 in order to increase power density.

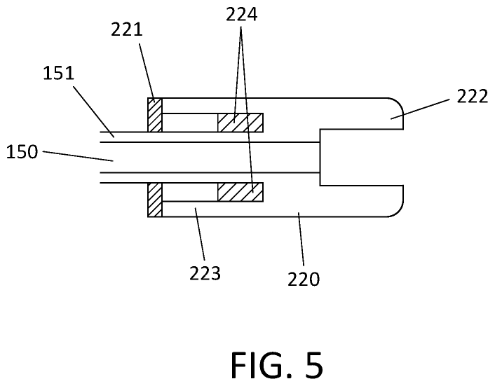

Finally, FIG. 5 shows an exemplary embodiment in which the reflective standoff sleeve 220 is further provided with an additional heat sink and/or reflector structure 221 at an upstream end of the sleeve, i.e., at the end of the sleeve 220 that is opposite the distal or output end 222. The heat sink and/or reflector structure 221 prevent energy from being transmitted back towards the scope, and may also be utilized with any of the exemplary embodiments shown in FIGS. 1-3, 4A, and 4B. Optionally, as illustrated in FIG. 1, the upstream section 223 of the sleeve may be spaced from the fiber cladding, with the space including a filler material 224 with a matched or higher refraction index than the cladding to further absorb or scatter energy. Also, the distal or output end 222 of the sleeve may be rounded to protect the scope during insertion of the fiber.

As with the reflective standoff sleeves of FIGS. 1 and 2, the materials of the sleeves shown in FIGS. 3, 4A, 4B, and 5 may be varied and may include silica glass, reflective metal, and sleeves with reflective coatings or inserts that may be welded or otherwise fixed to the fiber cladding.

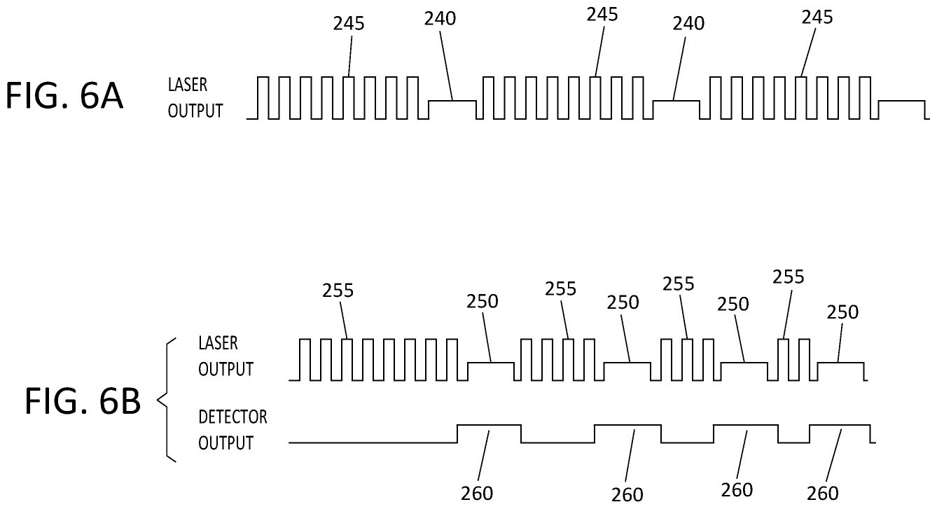

A problem common to the standoff sleeves shown in FIGS. 1-5, and to standoff sleeves in general, is that the destruction of stones and stone fragments creates suspended dust particles, which can build up on the inner diameter of the standoff sleeve, causing overheating and FEA despite a lack of contact between the fiber tip and the stone. To flush the suspended dust particles from the inside of the sleeve before they can accumulate, the laser output may be controlled in the manner illustrated in FIGS. 6A and 6B to add low power, long duration single or multiple pulses. The long duration pulses cause retro repulsion of the suspended dust particles to clear them from the interior of the sleeve.

As shown in FIG. 6A, for example, low frequency, low power pulses 240 may simply be added to a high frequency pulse train 245 at regular intervals. Alternatively, as shown in FIG. 6B, the low frequency, low power pulses 250 may be inserted into the laser output 255 whenever a stone sensing detector senses the presence of excessive radiation caused by dust particle buildup or FEA and outputs triggering pulses 260. The detector may utilize the stone sensing methods described in the above-cited copending U.S. patent application Ser. Nos. 15/992,609 and 17/400,380.

As an alternative to insertion of low frequency, lower power pulses at regular intervals, clearing of suspended dust particles can also be achieved by use of a single continuous background pulse or waveform, or by adding a pre-pulse for each or initiated therapeutic pulse. The dust particle clearing pulses can be created by modulation or appropriate control of the main therapeutic laser, or by a secondary laser.

Although preferred embodiments of the invention have been described in connection with the appended drawings, it will be appreciated that the description of the preferred embodiments is not intended to be limiting, and that modifications of the preferred embodiments may be made without departing from the scope of the invention, which should be limited solely by the appended claims.

For example, while the tapered fiber and metal, glass, or sapphire standoff sleeve illustrated herein are particularly adapted for use with Thulium Laser Fiber (TFL) lithotripsy systems, both the taper and the standoff sleeve may be used with lasers other than continuous wave Thulium lasers, including pulsed laser systems, and end-firing lasers for procedures other than laser lithotripsy. In addition, the materials of the standoff sleeve and any filler may be varied, as may the manner in which the standoff sleeve is fixed to the tapered section of the fiber. Still further, the dust particle buildup prevention method shown in FIGS. 6A and 6B is not limited to use with standoff sleeves of the type described in detail herein, but my be rather may be used with any structure situated at the tip of the fiber that has a surface subject to dust accumulation.

What is claimed is:

1. A standoff sleeve arrangement for a laser surgery optical fiber, comprising:
an optical fiber; and
a standoff sleeve having a reflective inner diameter and fixed to a region at an end of the optical fiber,
wherein the reflective standoff sleeve extends a predetermined distance beyond the end face of the fiber to prevent contact between the end face of the fiber and a tissue targeted by a laser exiting the fiber.

2. A standoff sleeve arrangement as claimed in claim 1, wherein the reflective standoff sleeve is made of silica glass or sapphire, or ceramic.

3. A standoff sleeve arrangement as claimed in claim 2, wherein the standoff sleeve is a silica glass tube that is index matched to, or has a refraction index higher than, a cladding material of the fiber.

4. A standoff sleeve arrangement as claimed in claim 1, wherein the reflective standoff sleeve has a reflectivity-enhancing coating or structure on the inner diameter.

5. A standoff sleeve arrangement as claimed in claim 1, wherein the reflective standoff sleeve is made of metal and is welded to the fiber.

6. A standoff sleeve arrangement as claimed in claim 5, wherein the reflective standoff sleeve has a reflectivity-enhancing coating on the inner diameter.

7. A standoff sleeve arrangement as claimed in claim 1, wherein the optical fiber is tapered to form a tapered section having a diameter that increases towards an end face of the fiber.

8. A standoff sleeve arrangement as claimed in claim 7, wherein the standoff sleeve is a silica glass sleeve, and further comprising a reinforcing filler material present in a space between the standoff sleeve and at least the tapered section of the fiber, wherein the filler material has an index of refraction that is matched to or higher than an index of refraction of a cladding of the fiber to absorb, transmit, or scatter energy present in the cladding and prevent the energy from propagating back through the fiber.

9. A standoff sleeve arrangement as claimed in claim 7, further comprising a silica tube positioned in a space between the standoff sleeve and at least the tapered section of the fiber, wherein the silica glass tube has an index of refraction that is matched to or higher than an index of refraction of a cladding of the fiber to absorb, transmit, or scatter energy present in the cladding and prevent the energy from propagating back through the fiber.

10. A standoff sleeve arrangement as claimed in claim 7, wherein the silica glass tube has a distal rounded end surface.

11. A standoff sleeve arrangement as claimed in claim 1, wherein the standoff sleeve is a silica glass sleeve, and further comprising a reinforcing filler material present in a space between the standoff sleeve and an end section of the fiber, wherein the filler material has an index of refraction that is matched to or higher than an index of refraction of a cladding of the fiber to absorb, transmit, or scatter energy present in the cladding and prevent the energy from propagating back through the fiber.

12. A standoff sleeve arrangement as claimed in claim 1, wherein the standoff sleeve is a silica glass sleeve, and further comprising a reinforcing structure positioned in a space between the silica glass sleeve and an end section of the fiber, wherein the reinforcing structure has an index of refraction that is matched to or higher than an index of refraction of the fiber cladding to absorb, transmit, or scatter energy present in cladding and prevent the energy from propagating back through the fiber.

13. A standoff sleeve arrangement as claimed in claim 12, wherein the reinforcing structure is a silica tube.

14. A standoff sleeve arrangement as claimed in claim 1, wherein the reflective standoff sleeve is welded to an end section of the fiber.

15. A standoff sleeve arrangement as claimed in claim 14, wherein the reflective standoff sleeve includes a reflective coating or structure on the inner diameter of the reflective standoff sleeve, the reflective coating or structure facilitating welding of the reflective standoff sleeve to the end section of the fiber.

16. A standoff sleeve arrangement as claimed in claim 1, wherein the reflective standoff sleeve comprises an ETFE or PTFE sleeve with a heat resistant reflective coating or structure on an inner diameter of the reflective standoff sleeve.

17. A standoff sleeve arrangement as claimed in claim 1, further comprising a heatsink or reflector positioned at an upstream end of the reflective standoff sleeve to prevent energy from being transmitted back towards a scope through which the optical fiber has been inserted.

18. A standoff sleeve arrangement as claimed in claim 1, wherein a distal end surface of the fiber is planar.

19. A standoff sleeve arrangement as claimed in claim 1, wherein a distal end surface of the fiber has a convex or lens shape to focus laser radiation exiting the fiber.

20. A standoff sleeve arrangement as claimed in claim 1, wherein the standoff sleeve arrangement is adapted for a laser having a wavelength of 1900 to 2200 nm.

21. A standoff sleeve arrangement as claimed in claim 20, wherein the laser is a Thulium Fiber Laser.

22. A standoff sleeve arrangement as claimed in claim 21, wherein the reflective standoff sleeve is a silica glass standoff sleeve.

23. A standoff sleeve arrangement as claimed in claim 22, wherein the standoff sleeve is a silica glass tube that is index matched to a cladding material of the fiber.

24. A standoff sleeve arrangement as claimed in claim 22, wherein the optical fiber is tapered to form a tapered section having a diameter that increases towards an end face of the fiber.

25. A standoff sleeve arrangement as claimed in claim 24, wherein a core diameter D1 of the fiber is approximately 150 μm, a diameter D2 of a distal end surface of the tapered section is 180 μm, the fiber has a numerical aperture (NA) of 0.22, the numerical aperture of the taper is 0.22×(D1/D2)=0.121. and a divergence output half angle θ of laser radiation exiting from the distal end surface is given by arcsin (0.121) or approximately 7°.

26. A standoff sleeve arrangement as claimed in claim 21, wherein the laser surgery optical fiber is adapted for use in laser lithotripsy procedures.

27. A standoff sleeve arrangement as claimed in claim 1, wherein the laser surgery optical fiber is adapted for use in laser lithotripsy procedures.

28. A standoff sleeve arrangement as claimed in claim 1, wherein a distal end of the reflective standoff sleeve is expanded to decrease an output power density of the laser exiting the fiber.

29. A standoff sleeve arrangement as claimed in claim 1, wherein a distal end of the reflective standoff sleeve is swaged down to increase an output power density of the laser exiting the fiber.

30. A method of clearing suspended dust particles from a standoff sleeve positioned at a tip of a laser lithotripsy fiber, comprising the steps of:
generating a pulse train made up of high frequency pulses for delivery through a laser lithotripsy fiber having a standoff sleeve at a treatment end of the fiber; and
inserting, into the pulse train, dust-particle-flushing pulses having a relatively lower power and longer duration than the high frequency pulses, to flush suspended dust particles from an interior of the standoff sleeve.

31. A method as claimed in claim 30, wherein the dust-particle-flushing pulses are inserted into the pulse train at regular intervals, as pre-pulses for initiated therapeutic pulses, or as a single continuous background pulse or waveform.

32. A method as claimed in claim 30, wherein the dust-particle-flushing pulses are inserted into the pulse train from a secondary laser.

33. A method as claimed in claim 30, wherein the dust-particle-flushing pulses are inserted into the pulse train in response to detection of dust particle buildup within the standoff sleeve or free electron absorption at the treatment end of the fiber.
